(19) United States
(12) Patent Application Publication
(10) Pub. No.: US 2026/0133154 A1
(43) Pub. Date: May 14, 2026

(54) ELECTROCHEMICAL SHOTGUN TAGGING ASSAY WITH CATALYTIC AMPLIFICATION

(71) Applicant: Oxford University Innovation Limited, Oxford (GB)

(72) Inventors: Jason Davis, Oxford (GB); Mohamed Sharafeldin, Dunedin (NZ)

(21) Appl. No.: 19/101,496

(22) PCT Filed: Jul. 21, 2023
(86) PCT No.: PCT/GB2023/051933

§ 371 Date: Feb. 5, 2025

(30) Foreign Application Priority Data
Aug. 8, 2022 (GB) 2211527.3

(51) Int. Cl. G01N 27/327; C12Q 1/28; G01N 33/543

(52) U.S. Cl. CPC G01N 27/3277; C12Q 1/28; G01N 33/5438; G01N 2333/908

(57) ABSTRACT

The present invention relates to the electrochemical sensing of proteins using shotgun biotin tagging of proteins prior to their interfacial immunocapture and enzyme tag recruitment.

The present invention relates to the electrochemical sensing of proteins using shotgun biotin tagging of proteins prior to their interfacial immunocapture and enzyme tag recruitment.

### BACKGROUND

Electrochemical sensors have underpinned significant advances in disease diagnostics, environmental monitoring, and food quality control. Within these, performance improvements (sensitivity and/or selectivity) have been sought via strategies such as engineering electrode surface characteristics (either by increasing surface area or conductance), decreasing the susceptibility of the electrode surface to interfering species (antifouling surface modification), and using amplifying labels to boost redox signals.

The latter has been the most commonly employed approach to increase signal to noise ratios and to maintain control over sensor dynamic range (for example, by modifying the number of enzyme tags per binding event). A diverse body of amplifying labels have been reported for electrochemical sensors, with enzymes such as horseradish peroxidase, alkaline phosphatase, and glucose oxidase being commonly used. A growing library of inorganic enzyme mimetics have also been proposed as potentially more “robust” alternatives to natural enzymes. The vast majority of such amplifying labels are anchored to the antigens captured on an electrode surface through a secondary, antigen-specific labelling process, thereby mandating the use of paired antibodies or aptamer probes which is reflected by increasing assay cost and time.

In contrast to the immense research directed towards developing new enzyme mimetics or polymeric enzyme labels, very few attempts have been directed towards the development of new enzyme (or enzyme mimetics) substrates. Horseradish peroxidase (HRP) is arguably the most commonly used enzyme for signal amplification by virtue of its low cost, high stability, good catalytic activity and substrate abundance. Peroxidase substrates have dominated developments in derived optical sensors, but only a few of these substrates have been applied to electrochemical sensors, where 3,3′,5,5′-Tetramethylbenzidine (TMB) is the most commonly used. The HRP-catalysed oxidation of TMB into oxidized TMB (where enzyme catalytic sites are recycled by solution phase hydrogen peroxide) has been employed in prior precipitate-based electrochemical sensors (with HRP labelled antibodies): see, for example, Timilsina et al., Adv. Healthc. Mater., 2021, n/a, 2102244, and Fanjul-Bolado et al., Anal. Bioanal. Chem., 2005, 382, 297-302. However, this rather crude approach can be problematic and generate false negative or false positive results and high levels of inter-assay variation; both soluble and insoluble oxidation products are generated as well as some uncontrolled dimerization of the TMB radicals, some of which may be removed during washing: see Zupançiçet al., Adv. Funct. Mater., 2021, 31, 2010638. The direct peroxidase activity on hydrogen peroxide can also be used in the generation of quantifiable signatures, either directly (peroxide voltammetry) or mediated. In all of these cases enzyme (target) specific presence arises from its coupling to secondary antibodies or nucleic acid probes, necessitating more steps, more variables, a greater assay time, and substantial cost.

It is well-established that HRP will catalytically activate tyramine in the presence of hydrogen peroxide to generate highly reactive tyramine radicals. This process has been applied, with dye-functionalized tyramine derivatives, to signal amplification in fluorescent imaging such as the tyramine signal amplification (TSA) or catalysed reporter deposition (CARD) methods. Integral in this is the covalent coupling of generated tyramine radicals to protein tyrosine moieties: see, for example, Clutter et al., Cytometry A, 2010, 77, 1020-1031 and Faget and T. S. Hnasko, in ELISA: Methods and Protocols, ed. R. Hnasko, Springer New York, New York, NY, 2015, DOI: 10.1007/978-1-4939-2742-5_16, pp. 161-172. This approach has also been applied for electrochemiluminescence (see, e.g., Cao et al., Sens. Actuators, B, 2021, 331, 129427) and surface-enhanced Raman spectroscopy (SERS) (see, e.g., Fu et al., Anal. Chem., 2018, 90, 13159-13162). The HRP-tyramine system has also been utilised within standard dual antibody sandwich electrochemical assays with additional complex further amplification, e.g. the use of heavily modified nanoparticles or additional polymerisation steps (see, e.g., Hou et al., Anal. Chem., 2014, 86, 8352-8358, Yuan et al., Anal. Chem., 2012, 84, 10737-10744, and CN108445063A).

In another recent development, described in Sharafeldin et al., Anal. Chem., 2022, 94, 2375-2382, shotgun (i.e., non-specific) tagging, with redox active tagging moieties, of ensemble proteins in clinically relevant media has been performed, prior to specific capture of redox-tagged, target protein molecules at antibody-modified electrodes. This facilitated a convenient voltammetric quantification of markers down to sub-pg/mL levels and across several orders of concentration. The platform is simple, generic, highly sensitive and (unlike the majority of the methods identified above) requires no secondary labeling/binding or amplification.

Nonetheless, there remains a continuing need for new electrochemical assay methods for sensing proteins, which combine properties such as high sensitivity, selectivity, reproducibility, scalability, ease of use, high speed, practical simplicity, and low cost into a single platform.

### SUMMARY OF THE INVENTION

The present inventors have now modified their previously developed faradaic shotgun approach (see Sharafeldin et al., supra) by shotgun tagging of ensemble proteins with biotin-comprising tagging molecules prior to target protein-specific immunocapture and facile labelling with amplifying enzymes. The system requires only a single bespoke binding event (e.g. antibody-antigen-based immunocapture) and capitalizes on further amplification of electrochemical signal using a peroxidase enzyme (e.g., HRP) substrate. Enzymatic turnover of tyramine-containing redox active molecules (e.g., ferrocene-tyramine, Fc-Ty) specifically showers electrode-confined proteins with a covalently tethered redox active material (e.g., ferrocene), generating specific electrochemical signal (and overcoming the drawbacks associated with the use of conventional TMB strategies where noncovalent adsorption is dominant). With Streptavidin-Poly(HRP (St-poly(HRP)), for instance, this assay format is capable of exceptional levels of sensitivity (down to fg/mL; attomoles of protein) in serum. Substrate incubation times can be tailored to allow a tuning of the assay dynamic range; extending it from few pg/mL to tens of ng/ml or proteins.

The present invention thus develops the previously developed faradaic shotgun by replacing the previous redox tagging moieties with biotin tagging moieties, followed, after target-specific surface capture, by facile amplification that leverages the well-established and simple biotin-(strept)avidin interaction together with the similarly well-established peroxidase/peroxide/tyramine catalytic reaction. The new system is simple, advantageously involving only one bespoke antibody-antigen (or antibody-antigen-like) component (namely the specific reaction between the target protein and surface-confined receptor) and otherwise well-established and often commercially available reagents, whilst nonetheless benefiting from a signal amplification that enables exceptional sensitivity and dynamic range. The present invention is also advantageous to any previous methods that utilise somewhat similar amplification techniques (e.g., based on the HRP/tyramine catalytic reaction), for instance in terms of process simplification, elimination of the need for a secondary bespoke antibody-antigen or similar component, and/or sensitivity and dynamic range achievable.

The present invention thus provides an electrochemical method of sensing target protein molecules, which method comprises:
(A) attaching tagging molecules to protein molecules in a carrier medium that may contain said target protein molecules, wherein the tagging molecules (i) each comprise a biotin moiety, and (ii) non-specifically attach to the protein molecules to produce protein molecules tagged with biotin;
(B) contacting the carrier medium with an electrode that comprises receptors that specifically bind to target protein molecules tagged with biotin;
(C) contacting the electrode with peroxidase-containing molecules that each comprise (i) a biotin-binding moiety and (ii) a peroxidase moiety;
(D) contacting the electrode with a peroxide and redox active molecules, wherein the redox active molecules each comprise (i) a tyramine moiety and (ii) a redox active moiety; and
(E) electrochemically determining whether target protein molecules are present in the carrier medium.

The present invention further provides ferrocene tyramide (Fc-Ty) of formula

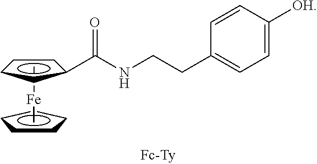

The present invention still further provides a kit for use in the method of the invention, which comprises:
(i) tagging molecules that each comprise a biotin moiety, and that non-specifically attach to protein molecules to produce protein molecules tagged with biotin;
(ii) peroxidase-containing molecules that each comprise a biotin-binding moiety and a peroxidase moiety; and
(iii) redox active molecules that each comprise a tyramine moiety and a redox active moiety.

Further preferred features and embodiments are described in the accompanying description and the appended claims.

### DESCRIPTION OF THE DRAWINGS

FIG. 1 is a schematic depiction of shotgun biotinylation of antigens using Biotin-LC-NHS followed by specific immune-recruitment at antibody coated electrodes. Captured targets are then incubated with streptavidin-Poly(HRP), washed and incubated with ferrocene-tyramine (Fc-Ty) in presence of hydrogen peroxide. The activated tyramine surface tethering to local tyrosine moieties of adjacent proteins generates a robust voltammetric signal that scales very sensitively with target concentration.

FIG. 2 depicts, with reference to Example 1, SPR analysis of successive incubation of increasing concentrations of (A) shotgun biotinylated α-Syn and (B) native (non-biotinylated) α-Syn on anti α-Syn modified SPR chips followed by injection of 100 ng/mL St-Poly(HRP). The inset shows the response of anti α-Syn modified SPR chips to α-Syn samples with increasing concentrations. Error bars represent one standard deviation over 5 sampling points across the SPR sensogram.

FIG. 3 depicts, with reference to Example 1, electrochemical signal after 10 min incubation of Fc-Ty on 10 pg/mL and 50 pg/mL HRP immobilized on 3 mm glassy carbon disc electrodes as compared to blank electrode. SWV were measured in 0.1 M KCLO4 after washing electrodes 3× with PBS-T20. Error bars represent one standard deviation (n=2).

FIG. 4 depicts, with reference to Example 1, cyclic voltammograms of screen-printed electrodes coated with 10 pg/mL HRP after exposure to Fc-Ty (20 mM) alone and to a solution containing 20 mM Fc-Ty and 25 mM hydrogen peroxide. The absence of redox peaks in absence of hydrogen peroxide highlights the critical role of H₂O₂ to enable the HRP-catalysed tyramine-mediated ferrocene deposition on the electrode surface.

FIG. 5 is a schematic depiction, with reference to Example 1, of an electrode array. The array is designed to fit into a bottomless standard 96 micro well-plate in a way that each well houses a 3 mm screen-printed carbon electrode, a screen-printed carbon counter electrode and Ag/AgCl reference electrode.

FIG. 6 depicts, with reference to Example 1, calibration of α-Syn using biotin shotgun tagging with Poly(HRP) generated covalent deposition of ferrocene after incubation with tyramine-ferrocene for (A) 5 min generating a dynamic range between 25 fg/mL to 400 pg/mL and (B) for 2 min to establish a dynamic range between 3.2 pg/mL and 10 ng/mL. Error bars represent one standard deviation of measurements from 4 different electrodes. Insets are representative square wave voltammograms of the tagged interface.

FIG. 7 depicts, with reference to Example 1, (A) specificity study on anti-α-Syn modified electrodes against large excesses of potentially interfering proteins. Error bars represent one standard deviation from measurements across 4 different electrodes. (B) reproducibility analyses generated by running the same concentration of α-Syn for 5 days over different electrode arrays. Each measurement represents an average across 6 individual electrodes (n=6). For each day, the bars represent, from left to right, measurements at concentrations of 128 fg/mL, 16 pg/mL and 80 pg/mL.

FIG. 8 depicts, with reference to Example 1, data from analysis of spiked 1% human serum containing different α-Syn concentrations. (A) bar chart comparison between spiked concentrations and found concentrations of α-Syn in a full range between 128 fg/mL and 400 pg/mL. (B) Same data presented in (A) but only covering the range between 128 fg/mL and 25 pg/mL. Error bars represent one standard deviation (n=4). For each panel and for each pair of bars shown, the left bar represents spiked α-Syn concentration and right bar recovered α-Syn concentration.

FIG. 9 depicts, with reference to Example 1, data from analysis of spiked 1% human serum with different α-Syn concentrations. (A) showing the correlation between spiked concentrations and found concentrations of α-Syn over a wide concentration range (between 128 fg/mL and 400 pg/mL). (B) Correlation between spiked and found α-Syn concentration over a narrow concentration range (between 128 fg/mL and 25 pg/mL). The regression coefficient R2=0.998 and slope of correlation is 1.01 indicating excellent agreement. Error bars represent standard deviations from repeats (n=4).

### DETAILED DESCRIPTION

Optional and preferred features of the present disclosure are now described. Any of the features described herein may be combined with any of the other features described herein, unless otherwise stated.

### Target (and Non-Target) Protein Molecules and Carrier Medium

The present disclosure is concerned with the sensing of protein molecules (e.g., determining the presence, or concentration, of such molecules in a carrier medium).

The term protein as used herein encompasses polymeric molecules that comprise a plurality of amino acid residues. Usually a protein molecule comprises at least 5, at least 10, at least 20, or, most commonly, at least 50 amino acid resides. However, unless expressly indicated there is no particular limitation on the lower or upper limit in the number of amino acids comprised by the protein molecules described herein (as long as it does not prevent specific binding of target protein molecules to the receptors comprised by the electrode, and as long as moieties for attaching the tagging molecules are present on the target protein molecules).

The protein molecules described herein can comprise any natural or non-natural amino acids. For example, a protein molecule may contain only α-amino acid residues, for example corresponding to natural α-amino acids. Alternatively, a protein molecule may additionally comprise one or more chemical modifications. For example, the chemical modification may correspond to a post-translation modification, which is a modification that occurs to a protein in vivo following its translation, such as an acylation (for example, an acetylation), an alkylation (for example, a methylation), an amidation, a biotinylation, a formylation, glycosylation, a glycation, a hydroxylation, an iodination, an oxidation, a sulfation or a phosphorylation. It is true that such post-translationally modified proteins still constitute a “protein” within the meaning of the present invention. For example, it is well established in the art that a glycoprotein (a protein that carries one or more oligosaccharide side chains) is a type of protein.

In the methods and kits of the present disclosure, protein molecules are contained in a carrier medium, typically a carrier liquid. The carrier liquid may be any liquid in which protein molecules can be suspended, dissolved, or dispersed. For example, the carrier liquid may comprise water. The carrier liquid may comprise or consist of a biological fluid. A biological fluid may be a fluid that has been obtained from a subject, which may be a human or an animal. In an embodiment, the carrier liquid comprises an undiluted biological fluid. An undiluted biological fluid in the present context is a biological fluid obtained from a subject, e.g. a human or animal, that has not been diluted with another liquid. The biological fluid may be selected from blood, urine, tears, saliva, sweat, and cerebrospinal fluid. Optionally, the carrier medium comprises a biological fluid obtained from a subject, e.g. a human or animal, and a diluent. The diluent may be added to the biological fluid after it has been obtained from the subject. The diluent may include a liquid medium, e.g. a liquid medium selected from water and an alcohol, e.g. an alcohol, e.g. ethanol. The carrier medium may further comprise a buffer. The buffer may comprise phosphate, saline, or other buffer components.

A carrier medium often contains protein molecules that are different from the target protein molecules. By “different” is meant the carrier medium contains (typically a plurality of) molecules of a protein that is chemically different (i.e. has a different chemical structure) from the target protein molecules, and does not merely refer to (physically) different molecules of a single protein. In other words, the carrier medium contains molecules of a protein that is (chemically/structurally) different from the target protein molecules. Such protein molecules differing from the target protein molecules can also be referred to herein as “non-target protein molecules”. By way of further illustration, in a method or kit directed to the sensing of α-Syn protein molecules (i.e. these being the target protein molecules), a carrier medium containing ten molecules of α-Syn protein but no other protein molecules is to be regarded as a carrier medium that contains only target protein molecules (i.e., it does not contain protein molecules that are different from the target protein molecules). In contrast, a carrier medium containing five molecules of α-Syn protein and five molecules of fibrinogen is to be regarded as a carrier medium that contains both target protein molecules and protein molecules that are different from the target protein molecules (i.e. non-target protein molecules).

In the present disclosure, a general reference to “protein molecules”, unless context dictates otherwise, includes both target protein molecules (if present) and non-target protein molecules (if present). In contrast, a specific reference to “target protein molecules” excludes non-target protein molecules and a specific reference to “non-target protein molecules” or “protein molecules that are different from the target protein molecules” excludes target protein molecules.

The carrier medium often comprises a mixture of at least 2, more preferably at least 4, and more preferably still at least 6 different proteins. For instance, the target protein molecules may be present in the mixture together with molecules of at least 1, more preferably at least 3, and more preferably still at least 5 different proteins. Such protein mixtures are, of course, common in samples of clinical interest, such as biological samples. Non-limiting examples of the different proteins that may be present in the mixture together with the target protein molecules include fibrinogen and human serum albumin. Another example of such a different protein is bovine serum albumin.

The target protein molecules are often molecules of clinical interest, for instance as biomarkers that may be useful for monitoring the health of a subject, the presence of a pathological condition, and/or the efficacy of an ongoing or concluded period of therapy for a particular pathological condition. The sensitive detection of biomarkers in physiological samples is of ever-growing interest in diagnosis. The present methods can be used in order to sensitively and selectively sense (and determine the concentration) of protein biomarkers, specifically by providing an electrode substrate that is functionalised with receptors that are capable of specifically binding to the biomarker of interest.

Examples of proteins of interest include proteins that are a biomarker of one or more of cardiovascular disease (CVD), neurodegeneration, cancer, myocardial infarction, diabetes, pathogenic infection and general trauma.

Strictly non-limiting examples of protein molecules that may be target protein molecules in the methods and kits described herein include dengue NS1 protein; angiotensin I converting enzyme (peptidyl-dipeptidase A); adiponectin; advanced glycosylation end product-specific receptor; alpha-2-HS-glycoprotein; angiogenin, ribonuclease, RNase A family, 5; apolipoprotein A-1; apolipoprotein B (including Ag(x) antigen); apolipoprotein E; BCL2-associated X protein; B-cell CLL/lymphoma 2; complement C3; chemokine (C—C motif) ligand 2; CD 14, soluble; CD 40, soluble; cdk5; pentraxin-related; cathepsin B; dipeptidyl peptidase IV; Epidermal growth factor; endoglin; Fas; fibrinogen; ferritin; growth hormone 1; alanine aminotransferase; hepatocyte growth factor; haptoglobin; heat shock 70 kDa protein 1 B; intercellular adhesion molecule 1; insulin-like growth factor 1 (somatomedin C); insulin-like growth factor 1 receptor; insulin-like growth factor binding protein 1; insulin-like growth factor binding protein 2; insulin-like growth factor-binding protein 3; interleukin 18; interleukin 2 receptor, alpha; interleukin 2 receptor, beta; interleukin 6 (interferon, beta 2); interleukin 6 receptor; interleukin 6 signal transducer (gp130, oncostatin M receptor); interleukin 8; activin A; leptin (obesity homolog, mouse); plasminogen activator, tissue; proopiomelanocortin (adrenocorticotropin/beta-lipotropin/alpha-melanocyte stimulating hormone/beta-melanocyte stimulating hormone/beta-endorphin); proinsulin; resistin; selectin e (endothelial adhesion molecule 1); selectin P (granule membrane protein 140 kDa, antigen CD62); serpin peptidase inhibitor, clade E (nexin, plasminogen activator inhibitor type 1), member 1; serum/glucocorticoid regulated kinase; sex hormone-binding globulin; transforming growth factor, beta 1 (Camurati-Engelmann disease); TIMP metallopeptidase inhibitor 2; tumor necrosis factor receptor superfamily, member 1 B; vascular cell adhesion molecule 1 (VCAM-1); vascular endothelial growth factor; Factor II, Factor V, Factor VIII, Factor IX, Factor XI, Factor XII, F/fibrin degradation products, thrombin-antithrombin III complex, fibrinogen, plasminogen, prothrombin, von Willebrand factor, D-dimer, alpha-synuclein (α-sync), C-reactive protein (CRP); Cardiac Troponin I (cTnI); Troponin T (TnT; TropT); glycated hemoglobin (HbAlc), insulin; TRIG; GPT; HSPA1 B; IGFBP2; LEP; ADIPOQ; CCL2; ENG; HP; IL2RA; SCp; SHBG; TIMP2; Covid-19 S protein; and bacterial biomarker proteins.

### Tagging Molecules

The tagging molecules are molecules that have the following features: (a) they each comprise a biotin moiety (i.e. each tagging molecule has a biotin moiety), and (b) they non-specifically attach to the protein molecules to produce protein molecules tagged with biotin.

With respect to feature (a), biotin moieties are very well known and widely used in the assay field. Biotin itself has the chemical formula

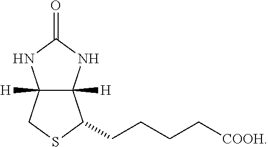 [SMILES: O=C(O)CCCC[C@@H]1SC[C@@H]2NC(=O)N[C@@H]21]

Biotin is well-known to bind specifically and strongly to biotin-binding moieties such as avidin and streptavidin proteins. A typical biotin moiety comprised by a tagging moiety of the disclosure similarly has the ability to bind specifically to a biotin-binding protein, for instance avidin or streptavidin.

An exemplary biotin moiety comprises at least the fused bicyclic ring system of biotin. The tagging moiety may be biotin itself (with the carboxylic acid group serving as a protein-binding moiety) or alternatively (and preferably) the carboxylic acid functionality of biotin itself may be leveraged to provide an alternative protein-binding moiety. In this alternative, the tagging moiety comprises both a biotin moiety and a protein-binding moiety (the latter as further described elsewhere herein). In this case, an exemplary biotin moiety is a moiety of formula

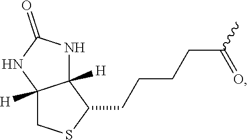

where the wavy line indicates the point of attachment to the protein-binding moiety (for instance, via an amide bond that includes the terminal carbonyl function shown in the formula).

For avoidance of doubt, the wording “ . . . each comprise a biotin moiety” is to be interpreted as requiring that at least one biotin moiety is comprised. It is not excluded that a tagging molecule comprises a plurality of biotin moieties in its structure, e.g. 2 or more, such as 2 to 5, for instance, 2, or 3, or 4, or 5. Conveniently though, each tagging molecule comprises 1 biotin moiety.

With respect to feature (b), by “non-specifically attach” is meant that the tagging moieties attach, e.g., bind, not only to a single specific type of protein, but attach, e.g. bind, to different types of protein; put another way, when contacted with a carrier medium containing at least two different types of protein, the tagging moieties attach, e.g. bind, to the different types of protein and not merely to (or predominantly to) one particular type of protein or sub-group of the types of protein. “Non-specifically” can be contrasted with “specifically”, particularly in the context of the specific binding exhibited by the receptors in the present disclosure (or indeed, between a biotin-moiety and a biotin-binding moiety such as (strept)avidin). Non-specific attachment/binding of proteins is, of course, well known in the art and its meaning would be readily understood.

In the present disclosure, a typical carrier medium contains protein molecules that are different from the target protein molecules (and the carrier medium may additionally contain target protein molecules). Typically, in such a carrier medium the tagging moieties attach to both: (i) target protein molecules, if present; and (ii) the protein molecules that are different from the target protein molecules. This attachment to both (i) and (ii) constitutes one non-limiting meaning for the expression “non-specifically attach(es) to protein molecules”. As already noted, “protein molecules” is hence a collective term; it includes both target protein molecules, if present in the carrier medium, and protein molecules that are different from the target protein molecules.

In one exemplary aspect of the invention, the protein molecules that are different from the target protein molecules comprise fibrinogen. In another exemplary aspect of the invention, the protein molecules that are different from the target protein molecules comprise human serum albumin. For instance, the protein molecules that are different from the target protein molecules may comprise both fibrinogen and human serum albumin. In a still further exemplary aspect of the invention, the protein molecules that are different from the target protein molecules comprise bovine serum albumin.

In a typical method of the disclosure, the step (A) of attaching tagging moieties to protein molecules in the carrier medium may comprise contacting the tagging moieties with the carrier medium and, thereby, reacting the tagging moieties with protein molecules therein to form protein molecules tagged with (and preferably covalently bound to) biotin.

When the carrier medium contains target protein molecules, step (A) produces target protein molecules tagged with (and preferably covalently bound to) biotin. When the carrier medium contains protein molecules that are different from the target protein molecules, step (A) produces protein molecules that: (i) are tagged with (and preferably covalently bound to) biotin; and (ii) are different from target protein molecules that are tagged with (and preferably covalently bound to) biotin.

The binding of the tagging moieties to protein molecules can be covalent or non-covalent in nature, but preferably is covalent. There is no particular limitation on the precise nature of the binding of the tagging moieties to protein molecules. For instance, the binding may occur through complementary functional groups on the tagging moieties, on the one hand, and the protein molecules, on the other hand. Hence, the tagging moieties preferably comprise a protein-binding portion.

It will be appreciated that particularly suitable strategies for binding the tagging moieties to protein molecules, given the desired non-specific nature of the binding, involve reactions with functional groups that are commonly present, and accessible for reaction, in a wide range of proteins (as usefully contrasted again with the specific binding exhibited by the receptors, which might typically involve, for instance, aptamers or antibodies (or fragments thereof) that recognise and bind, e.g. non-covalently, at least predominantly only to a specific, complementary protein).

For example, non-limiting, suitable binding strategies include those in which the tagging moieties feature, for instance as a protein-binding portion, arenediazonium salts, azlactone groups, vinyl sulfone groups, NHS-ester groups (NHS=N-hydroxysuccinimide) and isothiocyanate groups. Substantially any functional groups commonly found on proteins can be targeted (typical such groups being primary or second amines, such as at the termini of proteins and, especially, on lysine residue side chains, and the thiol groups of cysteine residue).

One exemplary binding strategy, for instance, utilizes an NHS-ester group on the tagging moieties for targeting amines (particularly primary amines such as lysine side chains) on protein molecules. The use of tagging moieties having an NHS-ester group can be particularly convenient owing to the resulting mild coupling conditions and high reaction rate to protein molecules.

In one purely non-limiting aspect of the disclosure, the term “non-specifically” means that the tagging moieties attach to the target protein molecules but also attach to an unrelated control protein (e.g. the “protein molecules that are different from the target protein molecules” described herein, including, but not limited to, fibrinogen, human serum albumin, or bovine serum albumin) such that the respective binding affinities are not substantially different. In one further entirely non-limiting example, the unrelated control protein is hen egg white lysozyme.

Preferably, particularly in the atypical situation where the non-specific binding is not covalent in nature, the tagging moieties attach to the target protein molecules with an affinity that is not more than 50, 25, 10, 5 or 2 times greater than (for instance that is substantially no greater than, i.e. that is substantially the same as) the affinity for a control protein (e.g., fibrinogen, human serum albumin, bovine serum albumin, or hen egg white lysozyme). Affinity may be determined by any means known in the art, such as by an affinity ELISA assay, a BIAcore assay, a kinetic method, and/or an equilibrium/solution method. Relative affinity for target protein molecules and control protein can also be measured using well-known competitive binding assays.

Preferably, particularly in the typical situation where the non-specific attachment is covalent in nature, under reaction conditions in which the tagging moieties attach (e.g., covalently bind) to the target protein molecules, the tagging moieties also attach (e.g., covalently bind) to a control protein (e.g., fibrinogen, human serum albumin, bovine serum albumin, or hen egg white lysozyme). Such reaction conditions include those typical when performing step (A) of the method of the present invention. For instance, one entirely non-limiting example of such reaction conditions are those in which the tagging moieties are contacted with an optionally buffered (e.g. MES buffer at pH 6.0) aqueous solution comprising both the said target protein molecules and the said control protein at 20° C.

Bearing in mind the above disclosure, one entirely non-limiting, exemplary and illustrative tagging molecule is a molecule of formula:

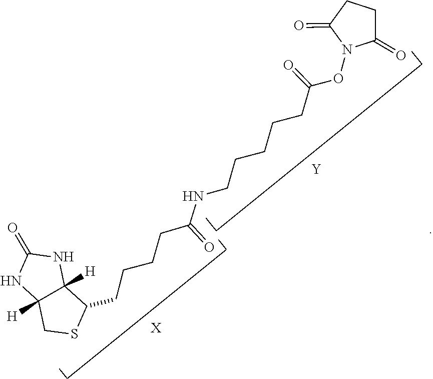

In this illustrative tagging molecule, portion X indicates a biotin moiety, while portion Y indicates a protein-binding portion that comprises an NHS-ester functional group.

Notably, tagging molecules of the type envisaged in the present invention are routine to manufacture and, in many cases, are commercially available as off-the-shelf products from companies such as Thermofisher, Abcam, and others. They are therefore easy and inexpensive to incorporate into commercial assay systems.

Whatever the precise structure of the protein molecules and tagging molecules, and the precise type of attachment (e.g., binding) that occurs between them, the physical result of performing step (A) of the method of the present invention is to produce protein molecules tagged with biotin. As will readily be understood, this arises because the biotin moiety comprised by the tagging molecules becomes attached (e.g., covalently attached) to the protein molecules. It will also be readily understood that the attachment may be either direct or indirect, i.e. via an intermediate chemical structure created by a reaction between complementary functional groups on the tagging moieties (e.g., located in a protein-binding portion thereof), on the one hand, and the protein molecules, on the other hand.

When the tagging molecules attach to target protein molecules they produce target protein molecules tagged with biotin. In contrast, when the tagging molecules attach to non-target protein molecules, they produce non-target protein molecules tagged with biotin. Unless context dictates otherwise, a generic reference to “protein molecules tagged with biotin” includes both target protein molecules tagged with biotin and non-target protein molecules tagged with biotin.

### Peroxidase-Containing Molecules

The peroxidase-containing molecules are molecules that have the following features: (a) they each comprise a biotin-binding moiety (i.e. each peroxidase-containing molecule has a biotin-binding moiety), and (b) they each comprise a peroxidase moiety (i.e. each peroxidase-containing molecule has a peroxidase moiety).

For avoidance of doubt, the wording “ . . . each comprise a biotin-binding moiety” is to be interpreted as requiring that at least one biotin-binding moiety is comprised. It is not excluded that a peroxidase-containing molecule comprises a plurality of biotin-binding moieties in its structure, e.g. 2 or more, such as 2 to 5, for instance, 2, or 3, or 4, or 5. Conveniently though, each peroxidase-containing molecule comprises 1 biotin-binding moiety.

Similarly, again for avoidance of doubt, the wording “ . . . each comprise a peroxidase moiety” is to be interpreted as requiring that at least one peroxidase moiety is comprised. It is not excluded that a peroxidase-containing molecule comprises a plurality of peroxidase moieties in its structure, e.g. 2 or. For instance, in preferred (though non-limiting) embodiments of the methods, and kits, of the present invention the peroxidase-containing molecule comprises a peroxidase polymer. Peroxidase polymers contain a plurality of peroxidase moieties, typically linked together via a polymer backbone, and are well known in the art and commercially available (for instance, poly-HRP).

With respect to feature (a) of the peroxidase-containing molecules, and as noted already, biotin is well-known to bind specifically and strongly to biotin-binding moieties such as avidin and streptavidin proteins. A typical biotin-binding moiety has the ability to bind specifically to biotin. In the context of the present invention, the biotin-binding moiety, in particular, has the ability to bind specifically to the present protein molecules tagged with biotin. “Specifically” has the same meaning as discussed elsewhere herein.

Most typically, the biotin-binding moiety is a biotin-binding protein. Such proteins are well known in the art, and examples thereof include avidin proteins, streptavidin proteins, and deglycosylated avidin (sometimes known as “Neutravidin”) proteins, all of which are well-known in the art and commercially available.

With respect to feature (b) of the peroxidase-containing molecules, the peroxidase moiety is an enzyme that is capable of acting on (i.e. utilising as a substrate) the peroxide component in the method (and kit) of the present invention and which is capable of using the redox active molecules (comprising tyramine moieties) as an electron donor in the reaction. As is well-known in the art, peroxidase enzymes catalyse the cleavage of the peroxide (—O—O—) linkage in peroxide compounds in the presence of hydrogen ions and a suitable electron donor. Many peroxidase enzymes are known in the art, acting on a wide range of peroxide compounds and being able to utilise a wide range of compounds as electron donors.

Preferred peroxidase enzymes are heme peroxidases, with a particularly preferred peroxidase enzyme being horseradish peroxidase (HRP). In one especially exemplary embodiment the peroxidase moiety is polymerised horseradish peroxidase, poly(HRP), which as already noted above is a commercially available, “off the shelf” product in which a plurality of HRP molecules are linked together via a polymer backbone.

In an exemplary embodiment, the peroxidase-containing molecule is streptavidin-poly(HRP). This substance is commercially available from companies such as Fitzgerald, for instance as Streptavidin Poly-HRP80 (average number of HRP monomer molecules of 80×5=400), Streptavidin Poly-HRP40 (average number of HRP monomer molecules of 40×5=200) and Streptavidin Poly-HRP20 (average number of HRP monomer molecules of 20×5=100).

Hence, suitable peroxidase-containing molecules for use in the present invention are also well-known in the art and, in many cases, commercially available as off-the-shelf products. Importantly, there is no requirement for creation of a bespoke peroxidase-containing molecule for use with a particular target protein. Rather, the same peroxidase-containing molecule is widely applicable for assays intended to detect any of a diverse range of target proteins.

### Peroxide

The peroxide component utilised in step (D) is a peroxide that is capable of functioning as a substrate for the peroxidase moieties comprised by the peroxidase-containing molecules (in conjunction with the redox active molecules as an electron donor). Usually the peroxide is hydrogen peroxide, e.g. when the peroxidase moieties are heme peroxidases such as HRP (for instance, when the peroxidase-containing molecules are streptavidin-poly(HRP) molecules).

### Redox Active Molecules

The redox active molecules are molecules that each comprise: (a) a tyramine moiety and (b) a redox active moiety.

With respect to feature (a), the tyramine moiety is typically a moiety having the following formula

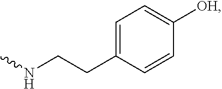

where the wavy line indicates the point of connection to the redox active moiety.

As previously noted, it is well-established that peroxidase enzymes such as HRP are able to catalytically activate tyramine in the presence of peroxides such as hydrogen peroxide to generate highly reactive tyramine radicals (a process in which the tyramine moiety thereby acts as an electron donor in a catalytic reaction on the peroxide). The present invention leverages this procedure to instead produce highly reactive radicals from the redox active molecules, namely ones that comprise the original redox active moiety component alongside an (oxidised) tyramine radical moiety component. These radicals react rapidly with protein tyrosine residues present in the immediate environment in which the radicals have been created: hence most typically on the (captured) target protein, the receptor, or other electrode-confined proteins. The result is a rapid build-up of redox active moieties on the electrode and drastic amplification of the resulting electrochemical response of the electrode. The amplification only occurs, however, when target protein molecules have been captured on the surface, because this is a pre-requisite for the supply thereto of the peroxidase moieties and hence the triggering of the catalytic reaction.

With respect to feature (b), by “redox active” means that the redox active moieties are capable of gaining or losing electrons (displaying redox activity) in order to change redox state. The redox active moieties, when confined in the vicinity of the electrode surface (e.g., as a result of binding mediated by the tyramine moiety, as further described elsewhere herein), modulate the electrochemical signal that is obtained in the electrochemical determination step (E) of the disclosed method, this modulation being a consequence of their redox activity. This modulation of electrochemical signal is therefore correlated to the absence, presence and/or concentration of target protein molecules in the carrier medium, in view of the fact that target protein molecule capture by the receptors is a pre-requisite to the subsequent capture of peroxidase and in turn generation of the highly reactive tyramine radicals that cause proliferation of redox active moieties captured on the electrode surface.

There is no particular limitation on the structure of the redox active moieties beyond that they exhibit the relevant redox activity. For instance, the redox active moieties may comprise a metallic chemical complex comprising a transition metal such as Fe, Ru, Ti, V, Mn, Cr, Co, Ni, Nb or Mo. Further representative examples of suitable redox active species that may constitute a redox-active portion include osmium-based redox active groups, ferrocenes, quinones and porphyrins, including derivatives thereof (e.g., alkyl (e.g., C₁₋₆ alkyl) or acyl derivatives of ferrocene). Derivatives of quinones include p-benzoquinone and hydroquinone. Another currently preferred example of redox active moieties that may constitute a redox-active portion are those that comprise a redox active portion derived from methylene blue, e.g. containing a structure

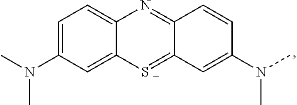

wherein the dashed line indicates a bond (to the remaining part of the redox active moiety).

One particularly preferred example of a redox active moiety is ferrocene. For instance, one exemplary redox active molecule is ferrocene tyramide (Fc-Ty), which has the formula

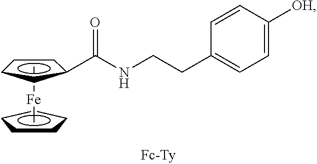

and which compound is itself also an embodiment of the present invention.

### Electrode

The electrode functions as the working electrode in an electrochemical system, specifically a system adapted for performing the methods described herein.

Typically the electrode comprises an electrically conductive substrate. This substrate may comprise any electrically conducting material. The substrate may comprise a metal or carbon. The metal may be a metal in elemental form or an alloy of a metal. Optionally, the whole of the substrate comprises a metal or carbon. The substrate may comprise a transition metal. The substrate may comprise a transition metal selected from any of groups 9 to 11 of the Periodic Table. The substrate may comprise a metal selected from, but not limited to, rhenium, iridium, palladium, platinum, copper, indium, rubidium, silver and gold. The substrate may comprise a metal selected from gold, silver and platinum. The substrate may comprise a carbon-containing material, which may be selected from edge plane pyrolytic graphite, basal plane pyrolytic graphite, glassy carbon, boron doped diamond, highly ordered pyrolytic graphite, carbon powder and carbon nanotubes.

In one embodiment, the substrate comprises gold, for example the substrate is a gold substrate. However, it is also possible for the substrate to comprise other materials and so, for instance, in other embodiments, the electrically conductive substrate is not a gold substrate. Non-limiting further examples of suitable electrically conductive substrates include carbon (e.g., graphene), platinum, silver, ruthenium oxide and indium tin oxide (ITO). The electrode can be a screen printed electrode (SPE) substrates (e.g. any of gold, carbon, platinum, silver, ruthenium oxide and ITO SPEs, for instance carbon, platinum, silver, ruthenium oxide and ITO SPEs). The electrode comprising the electrode substrate may, for instance, be one electrode in a multi-electrode array (i.e., a multiplex array).

The electrode surface (i.e., the substrate surface) may be planar, which includes a generally flat surface, e.g. without indentations, protrusions and pores. Such substrate surfaces can be readily prepared by techniques such as polishing with fine particles, e.g. spraying with fine particles, optionally in a sequence of steps where the size of the fine particles is decreased in each polishing step. The fine particles may, for example, comprise a carbon-based material (such as diamond) or a metal oxide (such as alumina), and/or may have particles with diameters of 10 μm or less, optionally 5 μm or less, optionally 3 μm or less, optionally 1 μm or less, optionally 0.5 μm or less, optionally 0.1 m or less. Following polishing, the substrate surface may be washed, e.g. ultrasonically, optionally in a suitable liquid medium, such as water, e.g. for a period of at least 1 minute, e.g. from about 1 minute to 10 minutes. Optionally, the substrate surface may be washed with an abrasive, e.g. acidic, solution, for example following the polishing and, if used, ultrasonic washing steps. The abrasive solution may comprise an inorganic acid, e.g. H₂SO₄, and/or a peroxide, e.g. H₂O₂, in a suitable liquid medium, e.g. water. Optionally, the substrates can be electrochemically polished, which may follow any steps involving one or more of polishing with fine particles, washing e.g. ultrasonically and/or using an abrasive solution. The electrochemical polishing may involve cycling between an upper and lower potential until a stable reduction peak is reached, e.g. an upper potential of 0.5 V or more, optionally 1 V or more, optionally 1.25 V or more, and a lower potential of 0.5 V or less, optionally 0.25 V or less, optionally 0.1 V or less.

### Receptors

The electrode comprises receptors that specifically bind to target protein molecules tagged with biotin.

As disclosed elsewhere herein, the electrode typically comprises an electrically conductive substrate. The electrode thus typically comprises both an electrically conductive substrate and receptors. Clearly, the receptors must be located such that they are able to contact the carrier medium, and hence any target protein molecules tagged with biotin (and hence bind thereto) when the method of the disclosure is performed. Additionally, the receptors must be stably associated to the electrically conductive substrate, such that they substantially do not detach from the electrically conductive substrate when the method of the disclosure is performed.

Otherwise, however, there is no particular limitation on the means by which receptors can be associated to an electrically conductive substrate. For instance, receptors may be covalently or non-covalently associated to an electrically conductive substrate, and associated either directly (e.g. via covalent or non-covalent bonding to the electrically conductive substrate itself) or indirectly (e.g. via covalent or non-covalent bonding to an intermediate layer, such as a non-fouling film, conductive polymer film, and/or self-assembled monolayer that is itself disposed on the electrically conductive substrate). As those skilled in the art would be well aware, methods for disposing receptors onto electrodes are well known in the art; any of these well-known and routine techniques can be used without limitation.

As applied herein and unless context dictates otherwise, “receptors” (in the plural) typically refers to a plurality of receptor molecules or moieties of a particular (common) chemical structure. For instance, anti-α-Syn antibody receptors (as utilized for illustrative purposes in the examples section) comprised by an electrode refers to a plurality of anti-α-Syn antibodies that are comprised by the electrode. The receptors (and by analogy each receptor molecule/moiety) are capable of specifically binding to target protein molecules tagged with biotin.

The term “specifically binds” and similar terms (“specifically binding”, “specifically bind”, etc.) in the context of binding between receptors and target protein molecules tagged with biotin means that the receptors bind to target protein molecules tagged with biotin with much greater affinity than they bind to an unrelated control protein. The concept of specific binding to proteins is very well known in the art; this term is therefore widely understood and those skilled in the art would have no difficulty, for a particular target protein, in identifying whether a particular receptor satisfies the requirement for having the recited capability of specifically binding thereto. Most commonly the specific binding is not covalent binding, but rather is non-covalent.

However, for avoidance of doubt, the distinction between binding to target protein molecules tagged with biotin and binding to an unrelated control protein typically excludes comparison with binding to (“native”) target protein molecules that have not been tagged with biotin (in other words, typically “unrelated control protein” does not include target protein molecules that have not been tagged with biotin). As those skilled in the art would of course appreciate, it is possible that a proportion of the protein molecules in the carrier medium may not in practice become tagged to biotin in step (A) of the disclosed methods. However, receptors described herein would often still exhibit specific binding to such (untagged) target protein molecules. Hence, specificity as relating to the binding properties of the receptors means specificity relative to different protein molecules, rather than the same protein molecules, but simply in this untagged state.

As with all binding agents and binding assays, those skilled in the art recognize that the various moieties to which a receptor should not substantially bind in order to be suitable would be exhaustive and impractical to list. Therefore, for receptors disclosed herein, the term “specifically binds” refers to the ability of the receptors to bind (e.g., non-covalently bind) to target protein molecules tagged with biotin with much greater affinity than they bind (e.g., non-covalently bind) to an unrelated control protein (including such an unrelated control protein that is tagged with biotin). In one entirely non-limiting example, the unrelated control protein is hen egg lysozyme. Another example is milk casein. Another example is fibrinogen. A further example is human serum albumin. A still further example is bovine serum albumin. A further example is any one of these proteins that is tagged with biotin. When carrying out the present disclosure in an embodiment where the carrier medium contains a particular protein that differs from the target protein molecules, then the unrelated control protein may preferably correspond to that particular protein (e.g., if the carrier medium contains fibrinogen and this is not the target protein, then this may be a preferred unrelated control protein; if the carrier medium contains human serum albumin and this is not the target protein, then this may be a preferred unrelated control protein; and if the carrier medium contains bovine serum albumin and this is not the target protein, then this may be a preferred unrelated control protein).

Preferably the receptors bind (e.g., non-covalently) to target protein molecules tagged with biotin with an affinity that is at least, 50, 100, 250, 500, 1000, or 10,000 times greater than the affinity for a control protein. The receptors may have a binding affinity for target protein molecules tagged with biotin of less than or equal to 1×10⁻¹⁰M, less than or equal to 1×10⁻¹¹M, or less than or equal to 1×10⁻¹² M. Affinity may be determined by any means known in the art, such as by an affinity ELISA assay, a BIAcore assay, a kinetic method, and/or an equilibrium/solution method. Relative affinity for target protein molecules tagged with biotin and control protein can also be measured using well-known competitive binding assays.

Examples of suitable receptors include antibodies, antibody fragments, nucleic acids, aptamers, oligosaccharides, peptides and proteins. Preferably, the receptor is selected from aptamers, antibodies, nucleic acids and peptides. More preferably the receptor is an aptamer or antibody, and most preferably an aptamer.

The antibody or the antibody fragment may be selected from one or more of the classes IgA, IgD, IgE, IgG and IgM. In a preferred embodiment, the antibody or antibody fragment is of the IgG type. The antibody or antibody fragment may be derived from a mammal, including, but not limited to, a mammal selected from a human, a mouse, a rat, a rabbit, a goat, a sheep, donkey and a horse. The aptamer may be selected from a peptide aptamer, a DNA aptamer and a RNA aptamer.

Clearly, the choice of receptor for a given electrode is determined by the identity of the target protein. For a particular target protein, a corresponding receptor that is capable of specifically binding thereto should be selected. As one illustrative, and purely illustrative example, if the target species is dengue NS1 protein (significant blood concentrations of which are associated with dengue virus infection), then the receptor should be a substance capable of specifically binding to dengue NS1 protein (or more precisely dengue NS1 protein tagged with biotin), such as a dengue NS1 antibody.

In a typical and preferred embodiment of the present invention, the tagging molecules non-specifically bind covalently to protein molecules, while the receptors specifically bind non-covalently to target protein molecules tagged with biotin.

As one further qualification, typically the receptors utilised do not specifically bind to biotin, e.g. they do not contain a biotin-binding moiety such a biotin-binding protein (e.g. avidin, streptavidin, and the like). It will be appreciated that this would be an undesirable characteristic of the receptors because it would facilitate their specific binding not only to the desired target protein molecules tagged with biotin, as produced in step (A) of the method of the invention, but also to any non-target protein molecules tagged with biotin that may also be produced in step (A) of the method of the invention.

### Electrochemical Method

The method of the disclosure in general comprises at least the steps (A), (B), (C), (D) and (E) as described herein.

As discussed in more detail elsewhere, in step (A) tagging moieties are attached to protein molecules in a carrier medium. The attaching is “non-specific”, meaning that the attaching is to both target protein molecules (if present in the carrier medium) and to non-target protein molecules (again, if present in the carrier medium). Carrying out step (A) thereby results in production of protein molecules tagged with biotin. If the carrier medium provided prior to the start of step (A) contains target protein molecules, then the carrier medium obtained at the end of step (A) contains at least some target protein molecules tagged with biotin.

In step (B), the carrier medium is contacted with the electrode that comprises the receptors. During step (B), target protein molecules tagged with biotin will specifically bind to the receptors and thereby become confined to the electrode. It is possible, and permissible within the bound of the invention, for at least some untagged target protein molecules also to specifically bind to the receptors (on the understanding that at least some tagged target protein molecules will also specifically bind to the receptors). However, neither tagged nor untagged non-target protein molecules will in substantial quantity bind to the receptors in view of the specificity of the receptors for the target protein over non-target proteins.

Preferably, step (A) of attaching the tagging moieties to protein molecules in a carrier medium is carried out before the carrier medium is contacted with the electrode in step (B) (i.e. step (A) precedes step (B)). For instance, this may advantageously reduce background signals associated with undesirable binding of tagging moieties to proteins on the electrode surface (e.g., antibodies, any blocking proteins, etc.).

After step (B) and before step (C), the electrode is preferably washed, for instance to remove any non-target proteins and/or tagged non-target proteins, as well as any other fouling material, that may be associated with the electrode after step (B) but which is not specifically bound to the electrode via the receptors. For instance, washing can be carried out using routine buffer solutions, such as phosphate buffer solution (PBS), optionally together a surfactant such as Polysorbate 20 (e.g., one suitable buffer solution being PBS together with Polysorbate 20). Hence, the method optionally includes a step (B2), between steps (B) and (C), of washing the electrode (e.g., to remove substances that are not specifically bound to the receptors).

In step (C), the electrode (e.g., receptors of which are bound to target molecules tagged with biotin) is contacted with the peroxidase-containing molecules. In this step, the biotin-binding moieties of the peroxidase-containing molecules engage in binding with the biotin tagged target molecules bound to the receptors. Hence, provided that at least some target molecules tagged with biotin are confined on the electrode (via the receptors), the result is that peroxidase moieties now become confined on the electrode, via the binding between the biotin and biotin-binding moieties. However, in the absence of any target molecules tagged with biotin confined on the electrode (e.g. if the carrier medium lacked any target protein), peroxidase moieties do not become confined on the electrode.

After step (C) and before step (D), the electrode is preferably washed, for instance to remove material that may be associated with the electrode after step (C) but which is not specifically bound to the electrode via the receptors. For instance, washing can be carried out using routine buffer solutions, such as phosphate buffer solution (PBS), optionally together a surfactant such as Polysorbate 20 (e.g., one suitable buffer solution being PBS together with Polysorbate 20). Hence, the method optionally includes a step (C2), between steps (C) and (D), of washing the electrode (e.g., to remove substances that are not specifically bound to the receptors).

In step (D), the electrode is contacted with the peroxide and the redox active molecules. In this step, peroxidase moieties confined on the electrode act on the peroxide via electron donation from the tyramine moieties of the redox active molecules, thereby generating highly reactive tyramine radical moieties on the redox active molecule which react with other electrode-confined components (notably tyrosine moieties on proteinaceous components such as the peroxidase itself, the target proteins, and/or the receptors) to (e.g. covalently) attach the redox active moieties to the electrode surface. Hence, when peroxidase moieties are confined on the electrode, which as noted above ultimately depends on the presence of target proteins in the starting carrier medium, the outcome of performing step (D) is to functionalise the surface with (a multitude) of redox active moieties (and which can therefore amplify the electrochemical signal obtained in step (E)).

Optionally, after step (D) and before step (E), the electrode is washed, for instance to remove material that may be associated with the electrode after step (D) but which is not specifically bound to the electrode via the receptors or otherwise strongly/irreversibly bound thereto (e.g. as a result of the reaction between tyramine radical moieties and suitable protein functional groups). For instance, washing can be carried out using routine buffer solutions, such as phosphate buffer solution (PBS), optionally together a surfactant such as Polysorbate 20 (e.g., one suitable buffer solution being PBS together with Polysorbate 20). Hence, the method optionally includes a step (D2), between steps (D) and (E2), of washing the electrode (e.g., to remove substances that are not specifically bound to the receptors or otherwise strongly/irreversibly bound to the electrode).

Finally, step (E) involves electrochemically determining whether target protein molecules are present in the carrier medium. Usually step (E) is carried out after all of steps (A) to (D) have been performed (and are complete).

There is no particular limitation on the precise nature of step (E). Rather, electrochemically determining whether target protein molecules are present in the carrier medium can be performed by using any electrochemical method suitable for determining whether a target species is associated with an electrode surface. As those skilled in the art would readily appreciate, the electrochemical determination functions by the modulation of electrochemical signal that results when the redox active moieties become confined on the electrode via the cascade of reactions described above in connection with steps (A) to (D).

Thus, if the carrier medium does contain target protein molecules then a particular experimental measurement will be obtained. On the other hand the measurement will be different if the carrier medium does not contain target protein molecules (as this will ultimately preclude the deposition of any significant amount of redox active moieties onto the electrode). Similarly, changes in the measurement will occur as the concentration of the target protein molecules in the carrier medium changes (typically the signal increasing as the concentration of the target protein molecules in the carrier medium increases). Conveniently, the changes as a function of target protein molecules concentration can be quantified by way of a series of control experiments performed using carrier medium containing known concentrations of target protein molecules, which enables preparation of a calibration curve showing the results as a function of concentration and which can therefore be applied to establish the concentration of target protein molecules in a test carrier medium from the electrochemical measurements made on that system.

Those skilled in the art would furthermore be well aware of the diverse range of electrochemical methods that are well known in the art for detecting such changes in the binding state of receptors comprised on electrodes. Such methods include, without any limitation, voltammetric methods such as differential pulse voltammetric (DPV) methods (including squarewave voltammetric, SWV, methods), in general pulsed voltammetric methods as well as cyclic voltammetry and impedance methods, including measurement of conductance and capacitance properties of the electrode comprising the receptors. For instance, therefore, step (E) can comprise electrochemically determining whether target protein molecules are present in the carrier medium by one or more of voltammetry (e.g. by DPV or SWV), by electrochemical impedance spectroscopy (EIS), by electrochemical capacitance spectroscopy, and the like. One merely exemplary, and non-limiting method is exemplified in Example 1.

Such electrochemical methods are also routinely quantitative in nature, i.e. they are capable of distinguishing the extent of binding to receptors on the electrode of target protein molecules, and hence the concentration of the target protein molecules in the carrier medium. Again, this principle is exemplified, in a non-limiting way, in Example 1. Consequently, in a preferred aspect of the disclosure, step (E) comprises determining the concentration of the target protein molecules in the carrier medium.

When carrying out the method of the disclosure it has been found that advantageous limits of detection (“LOD”) can be achieved. For instance, in preferred embodiments the limit of detection of the target protein molecules may be lower than 10 pg/mL, preferably lower than 1 pg/mL and more preferably still lower than 0.1 pg/mL.

It has also been found that the methods of the invention can achieve beneficially high sensitivity (i.e., distinguishable changes in measured response as a function of only small changes in target protein concentration).

### Microfluidics and Multiplexing

In one preferred aspect of the methods of the disclosure, the method is carried out in a microfluidic device. For instance, step (A) is performed in a microfluidic mixer and/or steps (B) to (E) are performed in a microfluidic well containing the electrode (and into which fluid from the microfluidic mixer can flow). Further, in the kit of the disclosure, the electrode may be adapted for use in a microfluidic well (e.g. having a size suitable for accommodation in a microfluidic well in a microfluidic electrochemical device).

The method may also be carried out in array format, including in a microfluidic array. Hence, the method may be carried out in an array format in which (at least) steps (B) to (E) are carried out at least two times using electrodes comprising receptors that bind specifically to different target protein molecules tagged with biotin, and thereby comprising electrochemically determining whether at least two different target protein molecules are present in the carrier medium (the first iteration of steps (B) to (E) being performed using an electrode that binds specifically to target proteins of a first type, the second iteration of steps (B) to (E) being performed using an electrode that binds specifically to target proteins of a second type, and so on). Alternatively, two or more electrodes comprising receptors that bind specifically to the same target protein molecules may be used, with the array set-up thereby providing contemporaneous repeats of detection measurements for the target protein.

In such a set-up, the method may comprise, in step (A), providing a tagged-carrier medium comprising protein molecules tagged with biotin, and which is followed in a step (A′) by dividing the tagged-carrier medium into two or more portions. Each portion is then, separately, subjected to the steps (B) to (E). Typically, for each portion p₁, p₂, p₃, etc. step (B) comprises contacting that portion with an electrode that comprises receptors r₁, r₂, r₃, etc. that bind specifically to biotin-tagged target protein molecules t₁, t₂, t₃, etc. As such, multiple target protein molecules t₁, t₂, t₃, etc. can be sensed from a single sample of carrier medium, optionally using a single device (such as a single microfluidic device that comprises a microfluidic mixer for preparing the tagged-carrier medium, which then forks into two or more microfluidic wells each containing an electrode comprising receptors r₁, r₂, r₃, etc.). This is an especially advantageous feature of the disclosure as it enables a multiplexed target quantification, i.e. the detection of different target proteins as an ensemble boosting the diagnostic value, without the need for different labels and from one single solution.

### Kit

Kits of the disclosure are suitable for, adapted for, and/or specifically designed for use in the methods of the present disclosure.

A typical kit of the present invention comprises at least: (i) tagging molecules as defined herein; (ii) peroxidase-containing molecules as defined herein; and (iii) redox active molecules as defined herein. Optionally, the kit further comprises (iv) an electrode as defined herein and/or (v) a peroxide as defined herein. The kit components may be provided in any form, for instance a form suitable for storage.

Optionally, the electrode may be provided in ready-to-use format, in a device such as a microfluidic device, such as for point-of-use applications. In other words, the kit may comprise an electrochemical device that comprises the electrode. The electrochemical device may be a microfluidic device, as described elsewhere herein.

The microfluidic device may comprise a microfluidic mixer for mixing a carrier medium with the tagging moieties. The microfluidic device may further comprise a microfluidic well containing the electrode.

The microfluidic device may be in array format. For instance, the microfluidic device may comprise a plurality of microfluidic wells, each containing an electrode that comprises receptors. The device may comprise at least two microfluidic wells, comprising at least: a first microfluidic well containing an electrode that binds specifically to target protein molecules of a first type; and a second microfluidic well containing an electrode that binds specifically to target protein molecules of a second type.

### Apparatus

In general, the methods of the present disclosure can be conducted on a suitable apparatus. This apparatus comprises an electrochemical spectrometer comprising the electrode as described herein (and which constitutes the working electrode of the spectrometer). The spectrometer typically further comprises a reference electrode and/or a counter electrode.

The apparatus optionally further comprises (a) a receiver configured to receive, from said electrochemical spectrometer, input data comprising measurements made on the electrode; and (b) a processor configured to convert said measurements into output data concerning the presence or absence of the target protein molecules in the carrier medium (e.g., the concentration of the target protein). The receiver and processor can be part of a computer. The functionality of the receiver and processor can be achieved by programming the computer to receive input data from the method and to process these data into the output data as described herein.

### EXAMPLES

### Example 1

In this example, a novel electrochemical protein quantitation based on the shotgun biotin tagging of proteins prior to their interfacial immunocapture and polymeric enzyme tag recruitment is demonstrated. The highly amplified faradaic signals generated from a ferrocene-tyramine adduct enable fg/mL (attomolar) levels of detection and span across 5 orders of magnitude dynamic range.

### SUMMARY

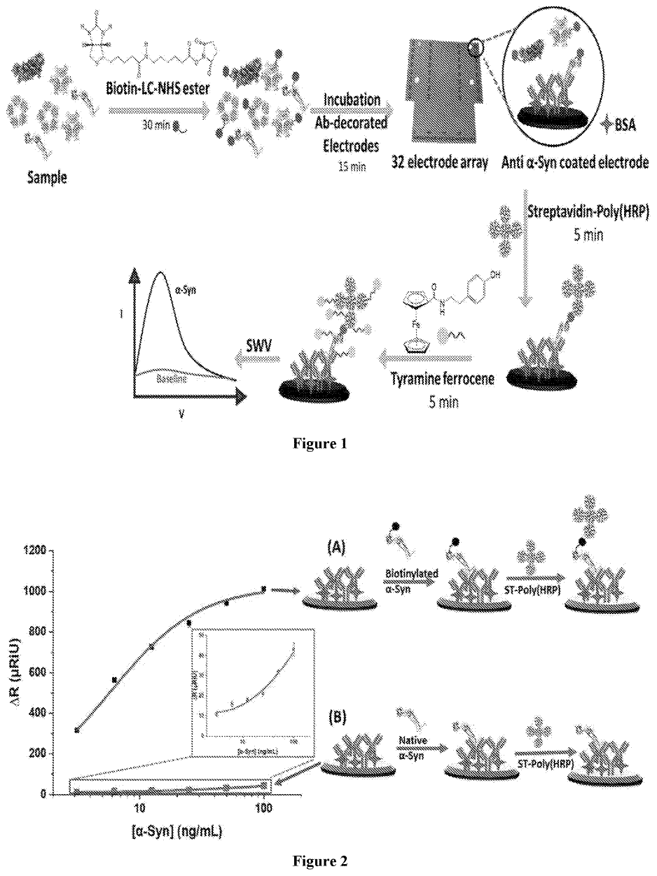

FIG. 1 provides a schematic depiction of the methodology utilised. Briefly, shotgun biotinylation of antigens was performed using Biotin-LC-NHS followed by specific immune-recruitment at antibody coated electrodes. Captured targets were then incubated with streptavidin-Poly(HRP), washed and incubated with ferrocene-tyramine (Fc-Ty) in presence of hydrogen peroxide. The activated tyramine surface tethering to local tyrosine moieties of adjacent proteins generated a robust voltammetric signal that scaled very sensitively with target concentration.

In this example, α-Syn was used as an illustrative target protein, with anti α-Syn as an illustrative receptor on the electrode surface, biotin-LC-NHS-ester as illustrative tagging molecules, streptavidin-poly(HRP) as illustrative peroxidase-containing molecules, H₂O₂ as an illustrative peroxide and tyramine-ferrocene as illustrative redox-active molecules.

Surface plasmon resonance (SPR) was initially employed to confirm target (α-Syn) biotinylation, the specificity of its recruitment at antibody (Ab) interfaces and then the specificity and efficacy of St-poly(HRP) recruitment. Biotin-LC-NHS was used to shotgun biotinylate proteins at a concentration of 200 μM (≈20 times the approximate molar concentration of total protein in 1% human serum), introducing 3-5 biotin tags per protein marker (See Materials and Methods below; see also Bradburne et al., Appl Environ Microbiol, 1993, 59, 663-668 and Leeman et al., Anal. Bioanal. Chem., 2018, 410, 4867-4873). While both biotinylated and non-biotinylated specific antigens are, of course, recruited onto the Ab-coated SPR chips, only the former showed a concentration-dependent response after exposure to St-Poly(HRP), confirming the specificity of HRP surface recruitment (FIG. 2).

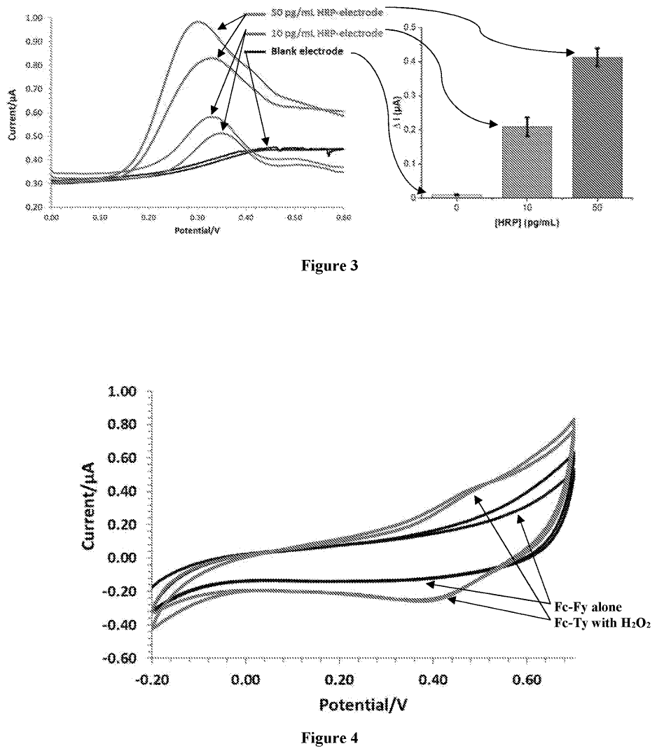

A simple coupling reaction (See Materials and Methods below; see also Hopman et al., J. Histochem. Cytochem., 1998, 46, 771-777) was used to synthesize a ferrocene-tyramine adduct (Fc-Ty) and its susceptibility to peroxidase activity tested. To confirm the so generated covalent tethering of Fc-Ty onto protein-functionalized electrodes, standard HRP initially used (diluted in 1% BSA) immobilized onto an electrode surface at different concentrations prior to incubation with Fc-Ty (8 mM in 1:1 (v/v) ethanol/water in 1 mM H₂O₂). Fc-Ty showed a predictably HRP concentration-dependent voltammetric signature (FIG. 3 and FIG. 4). In the presence of H₂O₂, HRP catalysed the conversion of Fc-Ty into highly reactive tyramide radicals that covalently coupled to local tyrosine moieties (that can be found on either HRP itself, adjacent BSA or indeed any local protein) (see also Kurisawa et al., J. Mater. Chem., 2010, 20, 5371-5375). For proteins which are electrode surface confined/captured, the electrochemical readout generated indicated a very sensitive quantification.

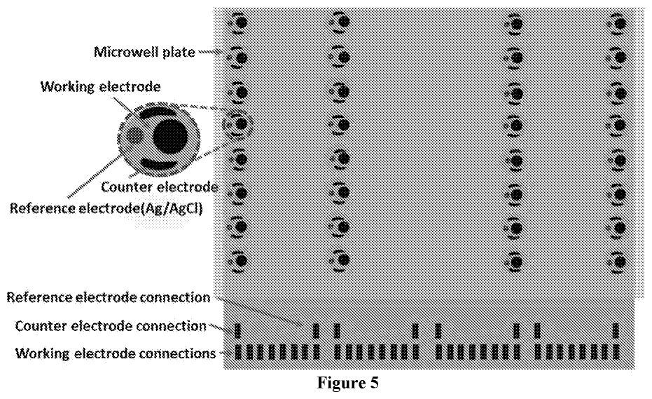

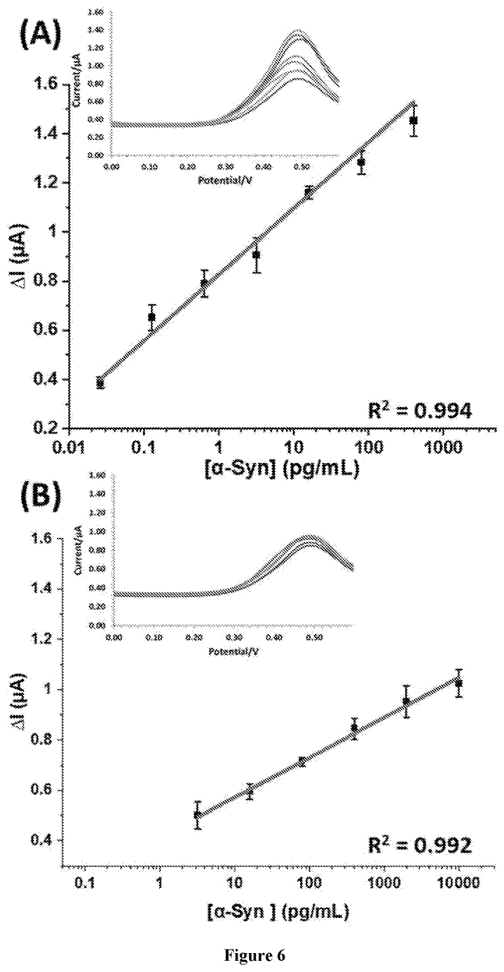

The shotgun biotinylation of α-Syn standards (spiked in 1% human serum) was then utilised for the development of an electrochemical sensor on screen printed carbon electrodes (FIG. 5). Physisorbed anti-α-Syn antibodies (on 32 electrodes housed in a conventional 96 micro-well plate) were allowed to capture biotinylated α-Syn from the shotgun tagged samples. Captured proteins were then labelled with St-Poly(HRP) and incubated for 2-5 minutes (depending on the desired dynamic range, see Materials and Methods below) with Fc-Ty substrate. The assays exhibited an ultra-low detection limit (down to 25 fg/mL) and a dynamic range spanning 5 orders of magnitude up to 400 pg/mL (FIG. 6A). The latter can be readily tuned simply by decreasing the incubation time of the Fc-Ty; for example, the dynamic range after 2 min incubation is between 3.2 pg/mL to 10 ng/mL α-Syn (FIG. 6B).

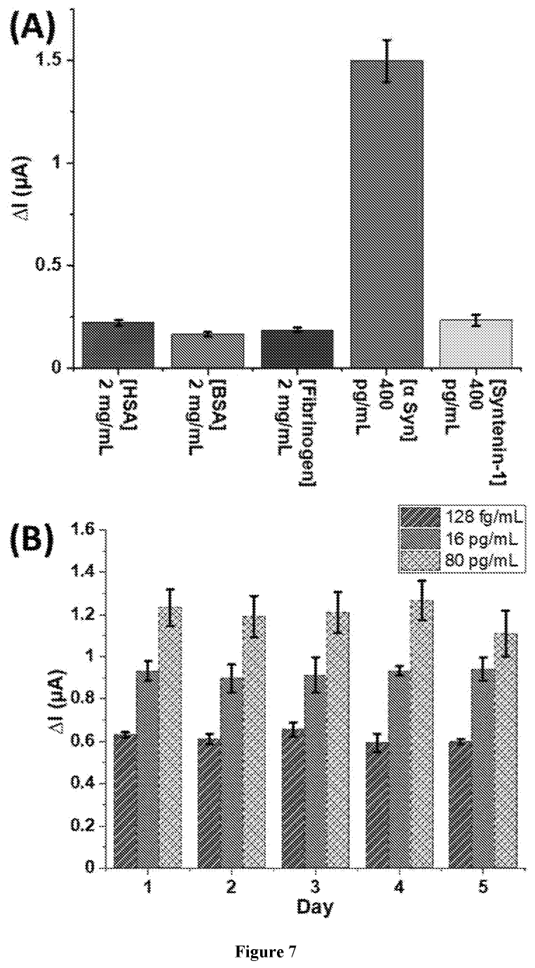

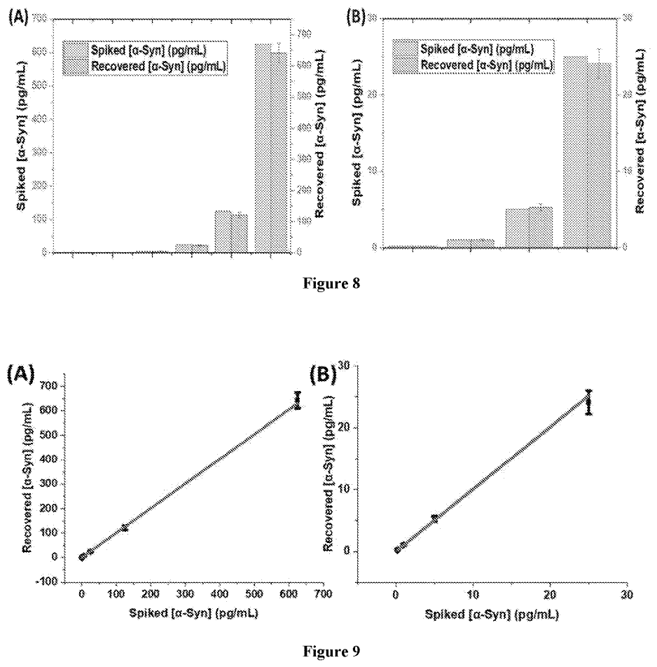

The electrochemical response to other common interfering proteins (which of course will also be shotgun tagged in solution) was assessed by exposing anti-α-Syn decorated electrodes to human serum albumin (HSA) (2 mg/mL), bovine serum albumin (BSA) (2 mg/mL), human fibrinogen (2 mg/mL), and human Syntenin-1 (Synt-1) (400 pg/mL). After incubation with St-poly(HRP), specific responses to α-Syn were observed with negligible (<10% of specific amperometric response to α-Syn) response to these large background levels: even at the simple antibody modified interfaces used here (FIG. 7A). The assays showed excellent reproducibility across different electrode arrays when repeated over a period of 5 days, with inter-array standard deviations <10% and inter-electrode variations (on the same array)<15% (FIG. 7B). An analysis of assay accuracy, as tested by spiked recovery experiments in neat human serum, demonstrated (see Table 1 below and FIG. 8) high precision with recoveries between 93-106% (FIG. 9). Table 1: Analyses of spiked samples in human serum compared to the spiked concentrations and calculated percentage recovery. Recoveries were between 97-106% indicating excellent assay accuracy.

| Spiked α-Syn Conc. | Recovered α-Syn | |
|---|---|---|
| [pg/mL] | [pg/mL] | Percent Recovery |
| 0.2 | 0.20 (±0.02) | 98.9 |
| 1 | 1.04 (±0.12) | 103.5 |
| 5 | 5.27 (±0.45) | 105.4 |
| 25 | 24.08 (±1.89) | 96.3 |
| 125 | 122.00 (±8.94) | 97.6 |
| 625 | 642.50 (±32.5) | 102.8 |

The sensory format developed here utilises a shotgun biotinylation and an interfacial immunocapture; the former enables the secondary tag to be a polymeric HRP. When this is coupled to an electrochemically active peroxidase substrate (Fc-Ty) extreme levels of assay sensitivity are accessible in assays that perform well in serum and avoid the drawbacks associated with physically adsorbed reagents (for example TMB), where nonspecific TMB adsorption can be highly problematic. The fact that these assays only use a primary antibody for selective labelled target recruitment directly reduces assay cost and time; most labelled assays require the introduction of a secondary antibody step before an additional labelling step; this requires 1-2 hrs and ˜20-30% of the total assay cost (USD 10-50/assay at 2022 costs). The introduced configuration is easily integrated into a screen-printed 32 electrode array housed in an ELISA-compatible 96-well plate enabling high throughput sample analysis within an ELISA-like workflow. The samples and reagents can be loaded manually or using a conventional automated ELISA analyser; allowing the rapid deployment into laboratories without the need for either significant hardware or special training.

The attomolar detection limits enable the detection of proteins that are expressed at levels that few assays can access. Usefully, the approach can also be tailored to the quantification of markers at much higher (ng/mL) levels and is readily extrapolated to any marker for which there is a well-established receptor. As tested here with spike recovery experiments in neat human serum, high levels of analytical precision are possible, with recoveries between 93-106%.

### CONCLUSIONS

In conclusion, a new sensor workflow capable of supporting protein quantitation down to few fg/mL concentration (attomoles), using a single recruiting antibody, has been introduced demonstrated. The assay employed common biotinylating reagent (Biotin-LC-NHS) to shotgun tag samples prior to specific interfacial capture. Exemplified here with α-Syn (as a Parkinson's-relevant model target biomarker), captured targets were then coupled to St-Poly(HRP), which catalysed the covalent tagging of local protein with redox addressable ferrocenes. The resulting voltammetric current reported on target analyte concentration with high specificity, selectivity and reproducibility.

### Materials and Methods

Materials and equipment: [2,5-Dioxo-1-[6-[5-(2-oxo-1,3,3a,4,6,6a-hexahydrothieno[3,4-d]imidazol-4-yl) pentanoylamino]hexanoyloxy]pyrrolidine-3-sulfonic acid sodium salt](Biotin-LC-NHS) and biotinylating reagent (Cat #ab145611) were purchased from Abcam© and used as provided. Mouse anti-Human α-Synuclein (α-Syn) capture antibody and recombinant human α-Synuclein standard antigen were purchased from R&D Systems© (Cat #DY1338). Streptavidin Poly-HRP80 conjugate was obtained from Fitzgerald©. Tyramine, fibrinogen, bovine serum albumin (BSA), human serum albumin (HSA), and human serum were purchased from Sigma-Aldrich©. N-Succinimidyl Ferrocenecarboxylate (Ferrocene-NHS) was bought from TCI©. All electrochemical measurements were carried out with a 3-electrode setup using a PalmSens 4 Multi-potentiostat powered by MultiTrace©. The SPR measurements were performed on Reichert DC7200 and data collected and analysed using integrated SPRAutolink software. Screen-printed 32 carbon electrode arrays were provided by Osler© diagnostics and fitted to ProPlate® bottomless microtiter 96 well-plate. Electrode arrays are designed such that each well houses a 3 mm screen printed carbon electrode, an Ag/AgCl reference electrode and a carbon counter electrode (Figure S1). Water used throughout was ultra-purified with a resistivity of 18.2 MΩ·cm (Milli-Q® Direct/Merck Millipore®).

SPR Study of solution phase shotgun biotinylation: SPR gold chips were cleaned by immersion in fresh Piranha solution (sulfuric acid and hydrogen peroxide 3:1 mixture) for 10 min, washed with water and ethanol and dried under nitrogen. The chip was then fitted into the sample holder of the Reichert SPR and allowed to equilibrate under running PBS buffer for 15 min. Anti α-Syn antibodies (20 pg/mL in PBS) were then injected and incubated for 1 hr for passive immobilization onto the chip surface. The chip was then washed and blocked by incubation with 1% BSA in PBS for 30 min. A series of concentrations of target protein, α-Syn, aliquoted in 1% BSA in PBS were incubated with 3.0 μM Biotin-LC-NHS for 30 min. The labelled (biotin-tagged) protein samples were injected onto the antibody modified SPR chip and incubated for 5 min and then washed. Subsequently, a 100 ng/mL Streptavidin Poly-HRP80 Conjugate-Poly(HRP) (St-Poly(HRP) was injected and incubated for 5 min, then washed. Similar procedures were repeated on Anti α-Syn modified chip with non-biotinylated protein samples.

Synthesis of Ferrocene tyramide (Fc-Ty): The synthesis of Fc-Ty was achieved by standard amide bond formation between ferrocene carboxylic acid NHS ester and tyramine as outlined in Scheme 1. As detailed below, this was carried out both on a smaller scale in situ (without product isolation, Approach 1) as well as on a larger scale including product isolation and characterisation (Approach 2). For practical reasons, we generally recommend the straight-forward synthesis and isolation of the purified Fc-Ty as a solid (Approach 2). However, it should be noted that the assay performance is identical in all cases.

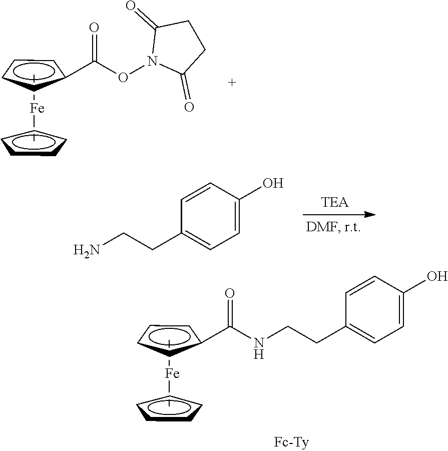

Scheme 1: Reaction pathway for the synthesis of tyramine-ferrocene by mixing Ferrocene succinimidyl ester with tyramine in DMF in basic environment.
Approach 1: In Situ Synthesis of Ferrocene tyramide (Fc-Ty):
Ferrocene carboxylic acid NHS ester (65 mg, 0.199 mmol, 1.1 equiv.) and tyramine (25 mg, 0.182 mmol, 1 equiv.) were dissolved in 5 mL anhydrous DMF. To this solution triethylamine (17 mg, 32 μL, 0.166 mmol, 0.91 equiv.) was added and reacted at room temperature for 2 h. This solution was used and stored for up to 8 weeks at 4° C.
Approach 2: Isolation and Synthesis of Ferrocene tyramide (Fc-Ty):
Ferrocene carboxylic acid NHS ester (200 mg, 0.612 mmol, 1 equiv.) and tyramine (84 mg, 0.612 mmol, 1 equiv.) were dissolved in 15 mL anhydrous DMF under N₂. To this solution triethylamine (62 mg, 85 μL, 0.612 mmol, 1 equiv.) was added and the solution was reacted under N₂ at room temperature overnight. 100 mL DCM were then added and the organic phase washed twice with water (100 mL) and once with brine (100 mL). The organic phase was then dried over MgSO₄ and reduced in vacuo. The product was purified by silica gel column chromatography (DCM/MeOH 97:3 (v/v)) to afford 159 mg (0.456 mmol, 75%) of Fc-Ty as an orange solid.

¹H NMR (400 MHz, DMSO) δ 9.15 (s, 1H), 7.79 (t, J=5.7 Hz, 1H), 7.19-6.95 (m, 2H), 6.83-6.59 (m, 2H), 4.75 (t, J=2.0 Hz, 2H), 4.31 (t, J=1.9 Hz, 2H), 4.09 (s, 5H), 3.35 (m, overlaps with solvent), 2.70 (t, J=7.4 Hz, 2H).

MS: (ESI+): 349.2 [M]+; (ESI−): 348.0 [M-H]−

Preparation of Fc-Ty Tagging Solution: The Fc-Ty reaction solution for covalent Fc-tagging of the HRP-modified interfaces was prepared by 5-fold dilution of a 40 mM DMF solution of Fc-Ty (either directly obtained by synthesis Approach 1 or prepared from solid Fc-Ty) with a 1:1 (v/v) mixture of EtOH/H₂O to give an overall concentration of 8 mM Fc-Ty.

Electrochemical shotgun assay: Electrode arrays were used as provided. Each array, consisting of 32 working screen-printed carbon electrodes, was washed with ethanol, dried under nitrogen and fitted into a bottomless 96 well-plate (ProPlate® microtiter plate). 50 μL of anti α-Syn antibodies (10 pg/mL) were added to each well and incubated at 4° C. overnight. Afterwards, the electrodes were washed, blocked by incubation with 100 μL of 1% BSA (in PBS) for 1 h. Protein samples (a series of concentrations of α-Syn) prepared in 1% BSA were biotinylated by incubation with 3.0 μM Biotin-LC-NHS for 30 min. When α-Syn samples were aliquoted in 1% human serum (diluted in PBS), 200 μM Biotin-LC-NHS was used to shotgun biotinylate proteins in the samples (assuming a total protein concentration of 80 mg/mL in neat human serum that is approximately 10 μM total protein for 1% human serum). Arrays were then washed and 50 μL of the labelled protein samples were added to each well in quadruplicates where they were incubated for 15 min (each sample is measured on 4 different electrodes/wells). After incubation, electrodes were washed with PBS-T20 (PBS with 0.05% Tween-20), incubated with 50 μL of 100 ng/mL St-Poly(HRP), washed and incubated with 50 μL of 8 mM Fc-Ty (in 1 mM H₂O₂) for 5 min to allow the HRP-catalysed tethering of ferrocene onto adjacent proteins. Finally, electrodes were washed with PBS-T20 and square wave voltammograms (SWV) were recorded in 0.1 M KClO₄ between 0.0 and 0.6 V vs Ag/AgCl reference electrode. The SWV pulse amplitude was 0.1 V with step increment of 0.01 V over 0.01 s intervals at 25.0 Hz frequency.

Specificity studies: Electrodes in screen printed carbon arrays functionalized with anti α-Syn antibodies were exposed to varying concentrations of common interfering proteins that were shotgun biotinylated in a procedure similar to abovementioned. After incubation, electrodes were washed with PBS-T20 and incubated with 50 μL of 100 ng/mL St-Poly(HRP), then washed with PBS-T20 and incubated with 50 μL of 8 mM Fc-Ty (in 1 mM H₂O₂). The SWV signals were measured and compared to those generated after incubating electrodes to 40 pg/mL α-Syn.

Spike recovery studies: A series of different concentrations of α-Syn were spiked into 1% human serum (HS) and shotgun biotinylated. These samples were then assayed using the same procedures described for electrochemical shotgun assay. The measured electrochemical signals were then used to back-calculate the found α-Syn concentration using the calibration data from recombinant α-Syn standards.

SPR Studies: The SPR studies were performed to confirm the shotgun biotinylation of proteins and the specificity of the ST-poly(HRP) binding to captured biotinylated α-Syn compared to the native α-Syn. FIG. 2 demonstrates a biotin-specific binding of the ST-poly(HRP) resulting in a significant SPR signal boost (compared to non-labelled α-Syn) due to the large molecular weight difference (ST-Poly(HRP) is approximately 1.8×10⁴ KDa and α-Syn is ≈14.5 KDa).

The electrochemical signal of HRP-catalysed deposition of Ferrocene-tyramine on HRP modified electrodes: To confirm the specificity of the tyramine-mediated ferrocene deposition, electrodes were decorated with different concentrations of HRP through simple physisorption on bare standard glassy carbon disc electrodes (3 mm). The ferrocene signal was absent in bare unmodified electrodes (after incubation with Fc-Ty) while HRP-modified electrodes showed concentration-dependent voltammetric signatures.

Spike recovery of α-Syn in 1% human serum: α-Syn spiked in 1% human serum (diluted in PBS) was analysed in 4 replicates for each concentration using the same assay protocol described for standards. The results showed an excellent correlation to spiked concentrations over a wide range from 128 fg/mL up to 400 pg/mL.

1. An electrochemical method of sensing target protein molecules, which method comprises:
(A) attaching tagging molecules to protein molecules in a carrier medium that may contain said target protein molecules, wherein the tagging molecules (i) each comprise a biotin moiety, and (ii) non-specifically attach to the protein molecules to produce protein molecules tagged with biotin;
(B) contacting the carrier medium with an electrode that comprises receptors that specifically bind to target protein molecules tagged with biotin;
(C) contacting the electrode with peroxidase-containing molecules that each comprise (i) a biotin-binding moiety and (ii) a peroxidase moiety;
(D) contacting the electrode with a peroxide and redox active molecules, wherein the redox active molecules each comprise (i) a tyramine moiety and (ii) a redox active moiety; and
(E) electrochemically determining whether target protein molecules are present in the carrier medium.

2. The method of claim 1, wherein step (C) comprises determining the concentration of the target protein molecules in the carrier medium.

3. The method of claim 1, wherein the carrier medium that may contain said target protein molecules is a biological sample.

4. The method of claim 1, wherein the carrier medium contains protein molecules that are different from the target protein molecules.

5. The method of claim 4, wherein the tagging molecules bind to both: (i) the target protein molecules; and (ii) the protein molecules that are different from the target protein molecules.

6. The method of claim 4, wherein the receptors: (i) specifically bind to target protein molecules tagged with biotin; and (ii) do not specifically bind to protein molecules that are different from the target protein molecules tagged with biotin.

7. The method of claim 1, wherein the receptors are selected from the group consisting of aptamers, antibodies, antibody fragments, oligosaccharides, peptides and proteins.

8. The method of claim 1, wherein the receptors are aptamers.

9. The method of claim 1, wherein the target protein molecules are selected from the group consisting of: Covid-19 S protein; dengue NS1 protein; angiotensin I converting enzyme (peptidyl-dipeptidase A); adiponectin; advanced glycosylation end product-specific receptor; alpha-2-HS-glycoprotein; angiogenin, ribonuclease, RNase A family, 5; apolipoprotein A-1; apolipoprotein B (including Ag(x) antigen); apolipoprotein E; BCL2-associated X protein; B-cell CLL/lymphoma 2; complement C3; chemokine (C—C motif) ligand 2; CD 14, soluble; CD 40, soluble; cdk5; pentraxin-related; cathepsin B; dipeptidyl peptidase IV; Epidermal growth factor; endoglin; Fas; fibrinogen; ferritin; growth hormone 1; alanine aminotransferase; hepatocyte growth factor; haptoglobin; heat shock 70 kDa protein 1 B; intercellular adhesion molecule 1; insulin-like growth factor 1 (somatomedin C); insulin-like growth factor 1 receptor; insulin-like growth factor binding protein 1; insulin-like growth factor binding protein 2; insulin-like growth factor-binding protein 3; interleukin 18; interleukin 2 receptor, alpha; interleukin 2 receptor, beta; interleukin 6 (interferon, beta 2); interleukin 6 receptor; interleukin 6 signal transducer (gp130, oncostatin M receptor); interleukin 8; activin A; leptin (obesity homolog, mouse); plasminogen activator, tissue; proopiomelanocortin (adrenocorticotropin/beta-lipotropin/alpha-melanocyte stimulating hormone/beta-melanocyte stimulating hormone/beta-endorphin); proinsulin; resistin; selectin e (endothelial adhesion molecule 1); selectin P (granule membrane protein 140 kDa, antigen CD62); serpin peptidase inhibitor, clade E (nexin, plasminogen activator inhibitor type 1), member 1; serum/glucocorticoid regulated kinase; sex hormone-binding globulin; transforming growth factor, beta 1 (Camurati-Engelmann disease); TIMP metallopeptidase inhibitor 2; tumor necrosis factor receptor superfamily, member 1 B; vascular cell adhesion molecule 1 (VCAM-1); vascular endothelial growth factor; Factor II, Factor V, Factor VIII, Factor IX, Factor XI, Factor XII, F/fibrin degradation products, thrombin-antithrombin III complex, fibrinogen, plasminogen, prothrombin, von Willebrand factor, D-dimer, alpha-synuclein (α-sync), C-reactive protein (CRP); Cardiac Troponin I (cTnI); Troponin T (TnT; TropT); glycated hemoglobin (HbAlc), insulin; TRIG; GPT; HSPA1 B; IGFBP2; LEP; ADIPOQ; CCL2; ENG; HP; IL2RA; SCp; SHBG; bacterial biomarker protein; and TIMP2.

10. The method of claim 1, wherein the tagging molecules comprise a biotin moiety and a protein-binding moiety.

11. The method of claim 1, wherein the tagging molecules comprise a protein-binding portion selected from an arenediazonium group, an azlactone group, a vinyl sulfone group, an NHS-ester group and an isothiocyanate group.

12. The method of claim 1, wherein the peroxidase-containing molecules comprise a biotin-binding moiety selected from the group consisting of streptavidin, avidin and deglycosylated avidin.

13. The method of claim 1, wherein the peroxidase-containing molecules comprise a horseradish peroxidase (HRP) moiety.

14. The method of claim 1, wherein the peroxidase-containing molecules comprise a polymerized horseradish peroxidase (poly(HRP)) moiety.

15. The method of claim 14, wherein the peroxidase-containing molecules are streptavidin-poly(HRP) molecules.

16. The method of claim 1, wherein the redox active molecules comprise a redox active moiety selected from: a group derived from methylene blue; a quinone; and a metallic chemical complex comprising a transition metal, wherein the transition metal is preferably Fe, Ru, Ti, V, Mn, Cr, Co, Ni, Nb, Mo or Os.

17. The method of claim 16, wherein the redox active moiety is a ferrocene moiety.

18. The method of claim 17, wherein the redox active molecules are ferrocene tyramide molecules (Fc-Ty) of formula

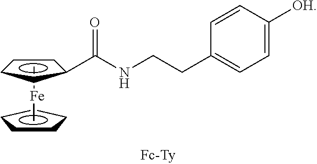

19. The method of claim 1, wherein the peroxide is hydrogen peroxide.

20. Ferrocene tyramide (Fc-Ty) of formula

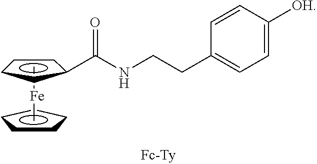

21. A kit for use in the method of claim 1, which comprises:
(i) tagging molecules, wherein the tagging molecules (i) each comprise a biotin moiety, and (ii) non-specifically attach to the protein molecules to produce protein molecules tagged with biotin;
(ii) peroxidase-containing molecules, wherein the peroxidase-containing molecules each comprise (i) a biotin-binding moiety and (ii) a peroxidase moiety; and
(iii) redox active molecules, wherein the redox active molecules each comprise (i) a tyramine moiety and (ii) a redox active moiety.

22. The kit of claim 21, which further comprises:
(iv) an electrode, wherein the electrode comprises receptors that specifically bind to target protein molecules tagged with biotin.

23. The kit of claim 21, which further comprises:
(v) a peroxide.
